(19) United States
(12) Patent Application Publication
(10) Pub. No.: US 2008/0178890 A1
(43) Pub. Date: Jul. 31, 2008

(54) METHOD AND APPARATUS FOR TRANSCERVICAL REVERSIBLE CORNUAL STERILIZATION

(75) Inventors: Phillip A. Townsend, Brookville, FL (US); Francis G. Moussy, Harefield (GB); Stuart Wilkinson, Zephyr Hills, FL (US)

Correspondence Address: DUANE MORRIS, LLP;IP DEPARTMENT, 30 SOUTH 17TH STREET, PHILADELPHIA, PA, 19103-4196, US

(73) Assignee: VACARE TEHNOLOGIES, LLC, Brooksville, FL (US)

(21) Appl. No.: 11/957,903

(22) Filed: Dec. 17, 2007

Related U.S. Application Data
(60) Provisional application No. 60/875,615, filed on Dec. 18, 2006.

(51) Int. Cl. A61F 6/06

(52) U.S. Cl. 128/831; 128/830

(57) ABSTRACT

A contraceptive device for transcervical reversible cornual sterilization includes an implant portion configured for implanting into the patient's uterine myometrium at a uterine cornu and circumscribing the fallopian tube's opening and a cap portion removably connected to the implant portion. The cap portion has an impermeable membrane substantially impermeable to the passage of reproductive cells, wherein when the implant portion is implanted into the uterine myometrium near the fallopian tube opening circumscribing the fallopian tube opening, the cap portion occludes the fallopian tube.

### CROSS-REFERENCE TO RELATED CASES

This is a U.S. non-provisional application of U.S. provisional patent application Ser. No. 60/875,615 filed on Dec. 18, 2006, the entire disclosure of which is incorporated herein by reference

### FIELD OF THE INVENTION

This disclosure relates to apparatus and method for providing permanent yet easily reversible sterilization of the human female by transcervical reversible cornual sterilization.

### BACKGROUND

Human sterilization refers to any procedure by which a person is rendered incapable of reproduction. Worldwide, two well tried and tested forms exist currently, tubal sterilization for women and vasectomy for men. Both forms are intended to be permanent and irreversible. Permanent tubal sterilization (or transabdominal tubal interruption) is currently the most common method of effective long-term contraception chosen by couples on a worldwide basis.

In the 1970's, the popularity of tubal sterilization and thus the number of surgical procedures performed increased dramatically in Europe, China, India, other parts of Asia and Latin America. In the United States, the number of tubal sterilization increased nearly fourfold—from approx. 200,000 in 1970 to about 700,000 in 1977. This increase was largely the result of development of two new surgical approaches, namely minilaparotomy and laparoscopy. These superceded the previous method of the 1920's to the 1950's of post partum laparotomy with the Pomeroy method of tubal occlusion.

In the United States in 1970 less than 1% of sterilizations were performed with a laparoscope, but by 1975, more than one third of the 550,000 women who had tubal sterilization had the procedure performed this way. The approach was facilitated in the early 1970's by the use of safe electro coagulation to permanently interrupt the tubal lumen, followed later in that decade by the development of spring loaded clips and Silastic rings for the same purpose.

In 1990 about 191 million married women of reproductive age used permanent sterilization (of themselves or of their spouses by vasectomy) for contraception. 169 million of these were in the developing countries and 22 million in developed countries. World-wide the ratio of female to male sterilization is 3 to 1. In the United States, sterilization is also the most commonly used form of contraception among married couples. The proportion of married couples who used tubal sterilization increased from 9% in 1973 to 28% in 1995. The increase in male sterilization (vasectomy) was much less dramatic, rising from 8% in 1973 to 11% in 1995.

The cumulative failure rate at 5 years for laparoscopic tubal sterilization in a major US study of procedures performed in the mid 1980's is quoted at 1 in 150. The likelihood of such a pregnancy being ectopic is extremely high. About 61% of the total when pregnancy occurs in the 4^{th} to 10^{th} years after tubal sterilization. The fatality rate associated with transabdominal tubal sterilization is in the order of 1 in 25,000. The most frequent serious immediate complications for laparoscopic tubal sterilization are associated with abdominal entry. These include bowel and major vessel injury in particular. For example in a study conducted in the United Kingdom, major vessel injury occurred in nine of 10,000 laparoscopies.

Modern hysteroscopy was introduced in the early 1970's as a method of visualizing the uterine cavity and uterotubal junctions. The idea of occluding this area by hysteroscopy was revived with considerable interest along with this developing technology. Examples of various transcervical and mainly hysteroscopic techniques used in the last 30+ years in an attempt to occlude the intramural portion of the fallopian tubes (interstitial oviducts) are: electrocoagulation and cryocoagulation; injection of chemicals into the uterine cavity for permanent closure; non-destructive occlusion by plastic preformed plugs; mechanical devices or tubal plugs that are placed in the proximal portion of the interstitial oviduct; and intratubal devices.

To date no successful, safe and efficient method of transcervical reversible tubal sterilization has been developed. The first and the only device approved (2002) by the United States Food and Drug Administration that is currently in use amongst the populus at the time of this patent application for permanent transcervical tubal sterilization, is the Essure System produced and marketed by the Conceptus corporation. It consists of a micro-insert, to obliterate the interstitial oviduct, a disposable delivery system, and a disposable split introducer. Because it is a transcervical tubal sterilization, the Essure System avoids the incision and the need for general anesthesia associated with transabdominal methods. However, this method again is not reversible.

By far the most common serious long-term risk associated with all forms of permanent sterilization, whether transabdominal or transcervical interruption of the fallopian tube lumen or vasectomy, is the patient's regret that this irreversible procedure was performed. In general, post sterilization regret increases over time. In the US Collaborative Review of Sterilization, the cumulative probability of regret went from 4% overall at 3 years to 13% at 14 years. At least five studies have identified young age at sterilization as the strongest predictor of later regret of sterilization. In the above study, the cumulative probability of expressing regret within 14 years was 20% for woman 30 years or younger versus 6% for those older than 30 years. Likewise, the 14 year cumulative probability of requesting information about reversal was 40% among women sterilized at 18 to 24 years compared to 10% for women over 30 years. Thus, a safe trsnscervical method of sterilization that is easily reversible is desired.

### SUMMARY

According to an embodiment, a contraceptive device for transcervical reversible cornual sterilization is disclosed. The contraceptive device comprises an implant portion configured for implanting into the patient's uterine myometrium at a uterine cornu and circumscribing the fallopian tube's opening and a cap portion removably connected to the implant portion. The cap portion has an impermeable membrane substantially impermeable to the passage of reproductive cells, wherein when the implant portion is implanted into the uterine myometrium near the fallopian tube opening circumscribing the fallopian tube opening, the cap portion occludes the fallopian tube.

According to another embodiment, a method for reversibly sterilizing a patient by occluding the patient's fallopian tube to prevent passage of reproductive cells is disclosed. The method comprises providing a contraceptive device in a collapsed configuration inside an inner lumen of a delivery cannula, the contraceptive device comprising an implant portion configured for implanting into the patient's uterine myometrium in a uterine cornu and circumscribing the fallopian tube's opening; and a cap portion removably connected to the implant portion, the cap portion having an impermeable membrane substantially impermeable to the passage of reproductive cells, wherein a guide wire extends through the contraceptive device. Next, the guide wire is advanced into a fallopian tube and the delivery cannula's distal end is advanced to the uterine cornu in proximity of a tubal ostium. The contraceptive device is then advanced out from a distal end of the delivery cannula and the implant portion of the contraceptive device is implanted by axially pushing the contraceptive device against the uterine cornu until the implant portion is substantially implanted into the patient's myometrium.

Unlike the current tubal sterilization techniques, sterilization achieved using the device of the present disclosure avoids any significant permanent interference with both structure and function of the uterus and fallopian tubes at the microscopic and molecular levels respectively following its removal. The contraceptive device of the present disclosure can be delivered to the appropriate site transcervically using either a hysteroscope or other existing well established technologies such as a cannula, thus avoiding an abdominal incision and surgical procedure within the peritoneal cavity that is common to all currently employed methods of tubal sterilization except for the Essure transcervical system. The contraceptive device of the present disclosure in contrast to all current tubal sterilization techniques, provides permanent yet easily reversible cornual sterilization.

### BRIEF DESCRIPTION OF THE DRAWINGS

A fuller understanding of the nature and objects of the present invention will become apparent upon consideration of the following detailed description, taken in connection with the accompanying drawings, wherein:

FIG. 1 illustrates the uterine and tubal anatomy in which the contraceptive devices disclosed herein are shown in place;

FIGS. 2-3 illustrate an embodiment of the contraceptive device of the present disclosure;

FIGS. 4-5 illustrate another embodiment of the contraceptive device of the present disclosure;

FIGS. 6-14 illustrate an example of the implantation procedure for the embodiment of the contraceptive device of FIGS. 2-3;

FIGS. 15-16 illustrate an example of the procedure for reversing the sterilization achieved with the contraceptive device of the present disclosure; and

FIG. 17 illustrates an example of a detailed view of the metal mesh frame work for the contraceptive device 100 of FIGS. 2-3.

All drawings are illustrated schematically and the structures illustrated therein are not to scale.

### DETAILED DESCRIPTION

This description of the preferred embodiments is intended to be read in connection with the accompanying drawings, which are to be considered part of the entire written description of this invention. In the description, relative terms such as “lower,” “upper,” “horizontal,” “vertical,”, “above,” “below,” “up,” “down,” “top” and “bottom” as well as derivative thereof (e.g., “horizontally,” “downwardly,” “upwardly,” etc.) should be construed to refer to the orientation as then described or as shown in the drawing under discussion. These relative terms are for convenience of description and do not require that the apparatus be constructed or operated in a particular orientation. Terms such as “connected,” “connecting,” “attached,” “attaching,” “joined,” and “joining” are used interchangeably and refer to one structure or surface being secured to another structure or surface or integrally fabricated in one piece, unless expressly described otherwise.

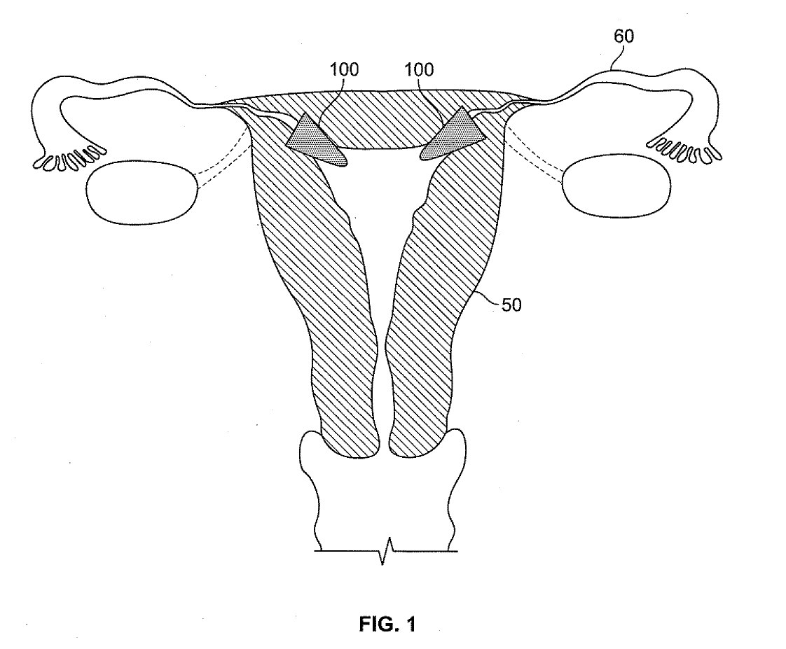

FIG. 1 illustrates the contraceptive devices 100 of the present disclosure implanted in the fallopian tube ostium of a uterus 50. As will be discussed in detail below, the contraceptive device 100 is transcervically inserted into the uterus 50 and implanted at the uterine opening of the fallopian tube 60 to block the passage of ovum from the fallopian tube 60.

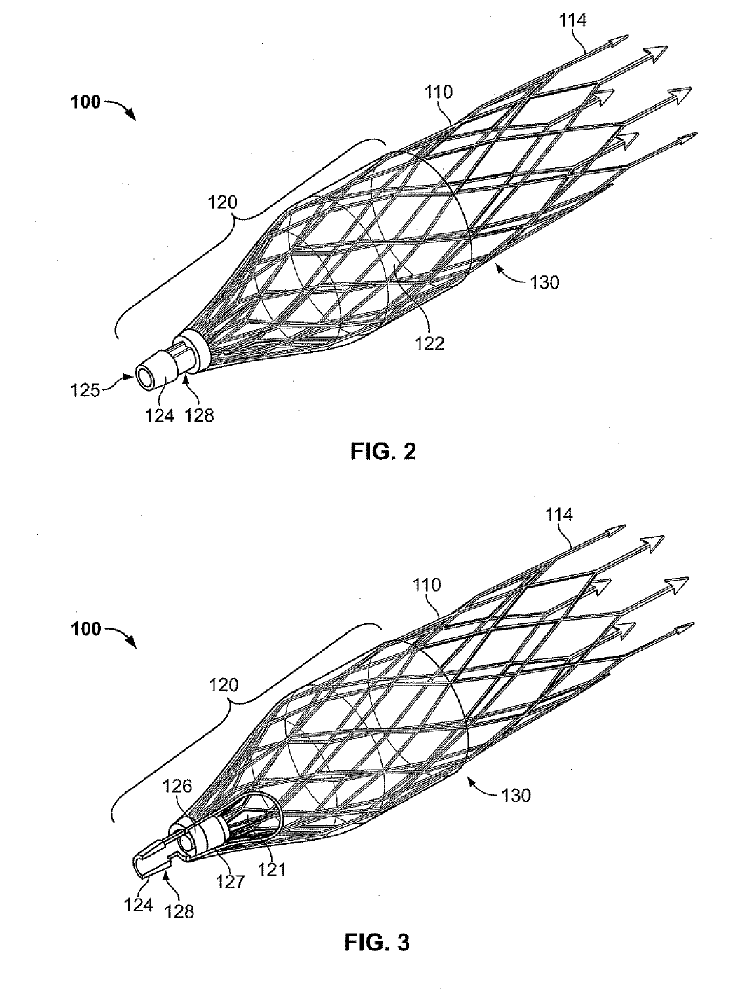

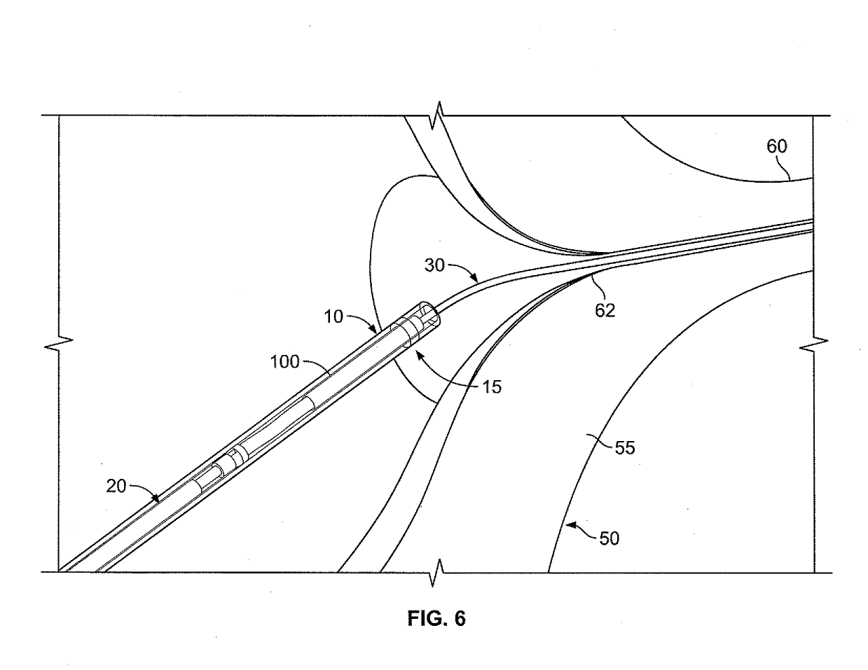

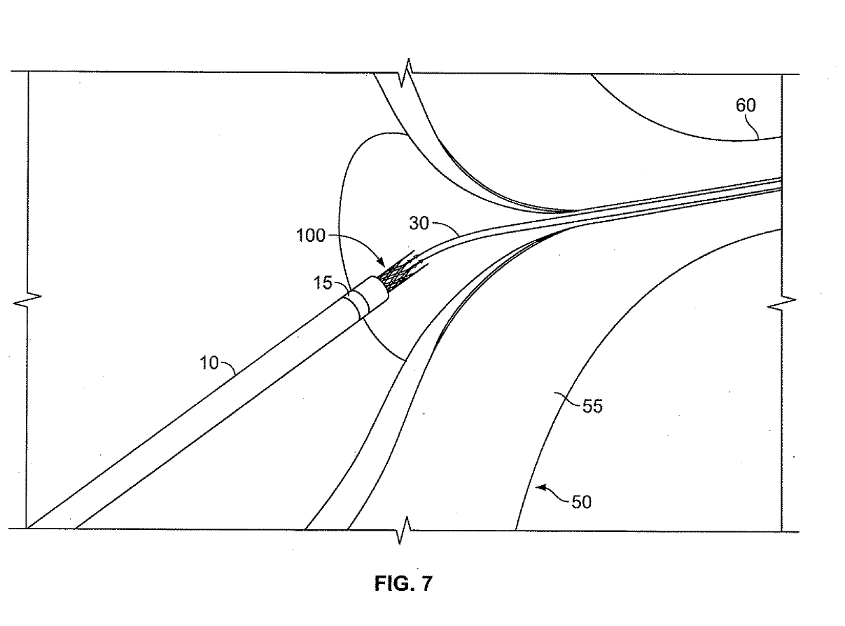

Referring to FIGS. 2-3, in one embodiment, contraceptive device 100 comprises a removable cap portion 120 and an implant portion 110. Both the implant portion 110 and the cap portion 120 are formed of self-expandable metal mesh structure. In one preferred embodiment, the metal mesh structure is made from a shape-memory alloy such as super-elastic Nitinol. The use of such material allows the contraceptive device 100 to be delivered transcervically in a collapsed compact state provided in a delivery cannula 10 (see FIGS. 6-7). When the contraceptive device 100 is deployed out of the distal end of the delivery cannula 10 near the tubal ostium of a uterus, the shape-memory alloy mesh structure of the contraceptive device 100 self-expands into an implant configuration.

The self-expanding property of such mesh structure made from Nitinol is well-known in stent technology so will not be discussed in detail. Basically the shape-memory metal alloy provides the contraceptive device 100 with a thermal memory of a desired configuration. The shape-memory alloy mesh can be fabricated out of an alloy stock that is about 1/150 inches thick. In one embodiment, the metal mesh can be fabricated by laser cutting the mesh structure out of an alloy stock tube into a mesh having a substantially cylindrical shape. Then this substantially cylindrical metal mesh is shaped into the configuration of the contraceptive device 100 where the distal end forming the implant portion 110 is open and the proximal end forming the removable cap portion 120 is formed into a conical shape. Where Nitinol shape memory metal is used, a particular alloy whose martensitic to austenitic phase transition temperature is just below a human body temperature is preferred. The contraceptive device 100 is deformed into a collapsed compact configuration small enough to fit inside the lumen of the delivery cannula 10 at a temperature below the transition temperature. When the contraceptive device 100 is brought to human body temperature the Nitinol metal mesh structure will elastically return to the original shape which is the implanting configuration shown in FIGS. 2-3. But, while the contraceptive device 100 is inside the delivery cannula 10, it is prevented from expanding back to the original implanting configuration. When the contraceptive device 100 is pushed out of the cannula 10 for implanting inside the patient's uterus, the device springs back to its implanting configuration. The contraceptive device 100 can be fabricated and configured to go from about 1.4 mm in diameter in the collapsed state inside the delivery cannula 10 to a diameter of about 5 mm at its fully deployed implanting configuration.

In this embodiment, the removable cap portion 120 of the mesh structure of the device can be coated with a suitable expandible polymer membrane 122 such as PTFE (e.g. Teflon), polyurethane or the like. The membrane 122 is impermeable to ova and spermatozoa. When the contraceptive device 100 is implanted in the patient, the implant portion 110 of the contraceptive device will be imbedded in the myometrium and the implant portion 110 is not covered with the impermeable membrane 122 in order to allow tissue ingrowth through the metal mesh structure for permanent fixation of the contraceptive device.

On the other hand, the removable cap portion 120 of the contraceptive device 100 is substantially or completely covered with the impermeable membrane 122. The impermeable membrane 122 may be formed by coating or molding the appropriate polymer in resin form on to the removable cap portion 120 of the metal mesh structure so that the impermeable membrane 122 and the metal mesh structure form a unitary structure. Alternatively, the impermeable membrane 122 can be preformed into the frustoconical shape and then bonded to the metal mesh structure. The attachment may be achieved by an appropriate adhesive or by ultrasonic bonding.

When the contraceptive device 100 is implanted, although the implant portion is completely embedded in the myometrium of the uterus the removable cap portion 120 is mostly exposed. The removable cap portion 120 being covered with the impermeable membrane 122 provides an impermeable barrier between the tubal ostium (proximal endometrial canal opening) and the endometrial cavity and thus between ovum and spermatozoa. Because the contraceptive device 100 needs to be collapsed into a compact configuration small enough to be fitted inside the delivery cannula 10, the impermeable membrane 122 is preferably flexible and able to collapse into the compact configuration along with the metal mesh structure of the contraceptive device.

Continuing with FIGS. 2-3, the proximal end of the removable cap portion 120 converges in a substantially frustoconical shape when the contraceptive device 100 is in an expanded configuration (i.e. implanting configuration) with the tip of the conical cap portion 120 being provided with a tip 124. The tip 124 is configured and adapted to securely hold the impermeable membrane and the expandable metal mesh 121 of the cap portion 120. This may be achieved by the use of a locking ring 126 provided on the interior side of the cap portion 120. The locking ring 126 may be configured to couple with the tip 124 by a snap-fitting arrangement while capturing the proximal end of the impermeable membrane 122. The proximal end of the shape-memory metal mesh 121 can be affixed to the locking ring 126 by any suitable method. Alternatively, the impermeable membrane 122 and the expandable metal mesh 121 can be attached to the tip 124 by an adhesive. If the tip 124 is fabricated from a suitable polymeric material, the impermeable membrane and the expandable metal mesh 121 can be attached to the tip 124 by ultrasonic bonding. Regardless of what attachment method is used to attach the impermeable membrane 122 and the expandable metal mesh 121 to the tip 124, the attachment must produce an impermeable seal that is impermeable to ovum and spermatozoa.

Both the tip 124 and the locking ring 126 are cannulated to have a passageway 125 extending therethrough for receiving a guide wire 30 (see FIGS. 2, 3, 6-12). Because the purpose of the contraceptive device 100 is to provide an impermeable barrier between the ovum and the spermatozoa, this passage way 125 must be closed once the contraceptive device 100 is implanted. This can be achieved by a sealing member 127 that operates as a one-way valve provided at the locking ring 126 end or the tip 124 end. In the illustrated example, the sealing member 127 is provided and coupled to the locking ring 126. The sealing member 127 can be a one-way valve that allows the guide wire 30 to pass therethrough but when the guide wire 30 is removed, it seals itself and closes the passage 125. The one-way valve can be a mechanical bi-valve or other appropriate sealing device.

In a preferred embodiment, the tip 124 can be further configured with a ridge 128 or similar structure(s) to be easily grasped by a miniature grasper 20 (see FIGS. 6, 11 and 12) for the purpose of securely holding the device during the implanting and removal procedure. The ridge 128 of this embodiment can extend substantially around the circumference of the tip 124.

The miniature grasper 20 can be configured to have two or more prongs at its distal end for grasping the tip 124 of the contraceptive device 100. The grasper 20 will hold the contraceptive device 100 while pushing the contraceptive device into the myometrium tissue until the implant portion 110 is fully implanted into the myometrium. The grasper 20 has sufficiently small diameter to fit within the delivery cannula 10. Thus, in one embodiment, the grasper 20 can be pre-packaged within a delivery cannula 10 device along with the contraceptive device 100 and the guide wire 30. In one possible configuration, the contraceptive device 100, the guide wire 30 and the grasper 20 are situated within a delivery cannula 10 with the contraceptive device 100 near the distal end of the delivery cannula 10. The guide wire 30 can be provided within the delivery cannula 10 just behind the contraceptive device 100. The guide wire 30 could be advanced through the one-way valve 127 of the contraceptive device 100 before passage of the delivery cannula into the working channel of a hysteroscope. Alternatively, the guide wire 30 can be advanced after the distal end (the end with the contraceptive device 100) of the delivery cannula 10 has been advanced to the proximity of the tubal ostium. If necessary, a delivery sheath may be provided at the entrance to the working channel of the hysteroscope to accommodate the delivery cannula 10. In this configuration, the grasper 20 is configured and adapted to lie within the lumen of the delivery cannula 10 through the length of the delivery cannula 10 along with the guide wire 30 such that both the grasper 20 and the guide wire 30 can be independently advanced towards the distal end of the delivery cannula 10 or retracted back. To that end, the grasper 20 can have a cannulated structure with a lumen for receiving the guide wire 30 therethrough. Alternatively, the grasper 20 can be configured with a channel running the length of the grasper 20 so that the grasper 20 and the guide wire 30 can be placed within the lumen of the delivery cannula 10 side-by-side.

In another embodiment, the grasper 20 can comprise two or three prongs fused together and packaged to lie within the length of the delivery cannula 10 alongside the guide wire. The non-fused distal ends would grasp the tip 124 of the contraceptive device 100 and push it along the guide wire into the myometrium. This pronged grasper 20 would then be removed along with the guide wire 30. The guide wire 30 itself would be packaged within the delivery cannula 10 alongside the fused prongs with its tip lying just proximal to the one way valve 127 of the contraceptive device 100 ready to be advanced through it at the time of implantation.

Preferably, all components of the contraceptive device 100 are comprised of materials that are relatively biologically inert, durable and resistant to uterine fluids.

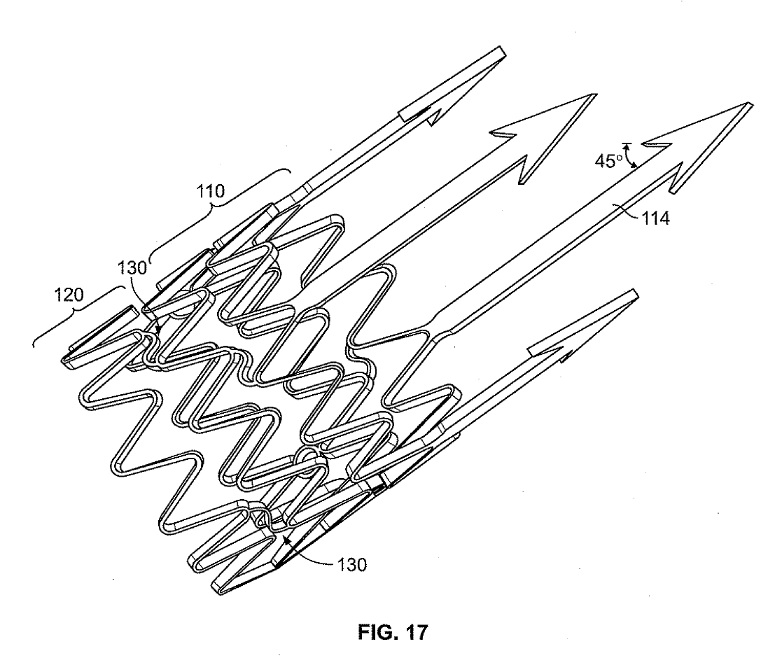

The distal end of the implant portion 110 is configured to be fixed in place in the myometrium close to, but not involving, the uterotubal junction. To enable the initial fixation of the contraceptive device 100 in the myometrium, the distal end of the implant portion 110 can be provided with barbs or barb-like structures 114. The barbs 114 can be fabricated as integral parts of the shape-memory metal mesh framework. In other words, the barbs 114 are designed into the metal mesh pattern and laser cut out of the same stock material. The barbs 114 are oriented in a longitudinal or axial direction, parallel to the longitudinal axis of the contraceptive device 100 so that the barbs can be driven straight into the myometrium without requiring any twisting motion or generating twisting motion. The distal ends of the barbs 114 have V-shaped tips angled at about 45° to firmly fix the device in place initially (see FIG. 17). The fixation of the contraceptive device 100 is further enhanced by fibrovascular ingrowth of the myometrium tissue through the metal mesh framework of the implant portion 110 that is not covered by the impermeable membrane 122.

Between the implant portion 110 and the cap portion 120 of the metal mesh comprising the contraceptive device 100 is a break-away zone 130. The break-away zone 130 is configured to break or sever when the removable cap portion 120 is deformed by laterally squeezing the cap portion 120. This feature allows the removable cap portion 120 to be removed after the implant procedure to allow reversal of the sterilization.

In one embodiment, the break-away zone 130 can be formed by the metal mesh structure having weakened joints along the metal mesh circumscribing the substantially tubular structure of the contraceptive device 100. Thus, similar to soda can tops being popped open, when the removable cap portion 120 is squeezed laterally, the deformation of the metal mesh structure stresses the weakened joints along the break-away zone 130 and snaps them off. The weakened joints of the metal mesh can be made by crimping or scribing the metal mesh wires after the mesh frame work is fabricated. Alternatively, the implant portion 110 and the removable cap portion 120 of the contraceptive device 100 can be fabricated as two separate pieces and then spot welded together in which the welded joints are of such strength to form the break-away zone 130. FIG. 17 shows a detailed view of an example of the metal mesh work for the implant portion 110, the removable cap portion 120 and the break-away zone 130. The break-away zone 130 in this example is configured to snap off when the cap portion 120 is deformed by squeezing.

In another embodiment, the implant portion 110 and the removable cap portion 120 are fabricated as two separate structures and then mechanically fitted together like pieces of a jig-saw puzzle. The mechanical fitting is configured to be sufficiently strong to keep the two portions together until they are separated by laterally squeezing the cap portion 120 in order to remove the cap portion 120.

Referring to FIGS. 6-13, the procedure of implanting the contraceptive device 100 according to an embodiment will be described. The contraceptive device 100 may be delivered to the implantation site and embedded therein by a number of presently existing and future technologies. In this example, the contraceptive device 100 is provided in its compact collapsed configuration inside a deliver cannula 10 with a guide wire 30 inserted through the contraceptive device 100. The cannula 10 is used to reach the implantation site transcervically. This can be done either by inserting the cannula 10 through a working channel of a hysteroscope (not shown) or inserting the cannula 10 directly through the cervical canal of the patient. Once the distal end of the cannula 10 is maneuvered to the implantation site, the guide wire 30 is inserted into the fallopian tube ostium 62 (see FIG. 6). The guide wire 30 may be lubricated or coated with a hydrophilic coating material to minimize any cell damage.

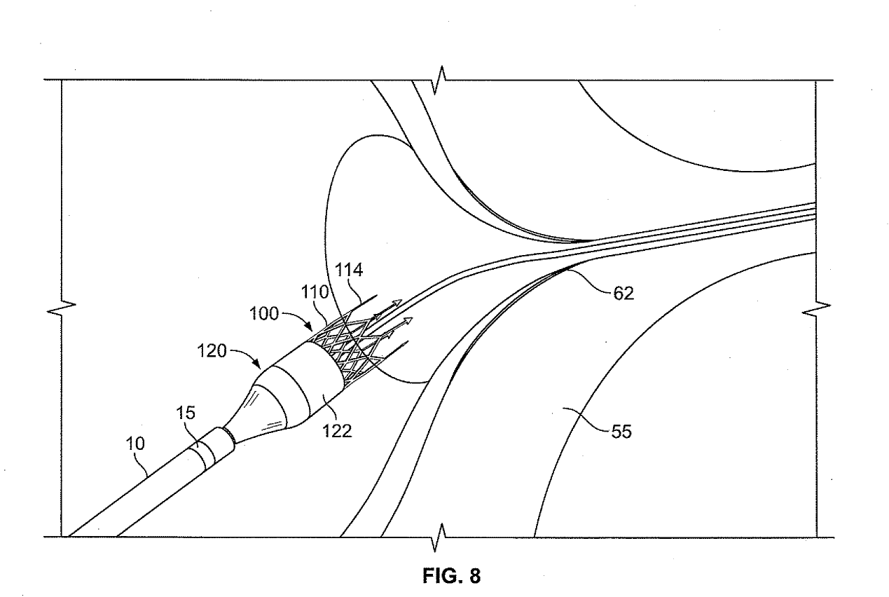

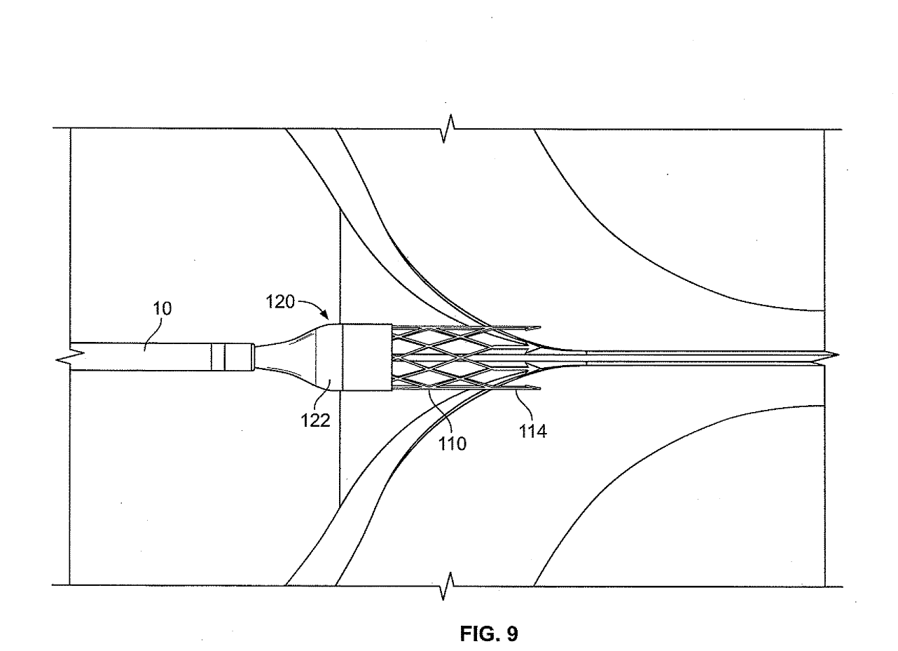

The contraceptive device 100 is then pushed towards to the distal end of the cannula 10 using the grasper 20 and out of the cannula 10 (see FIG. 7) Once fully deployed out of the cannula 10, the contraceptive device 100 self-expands into the implant configuration (see FIG. 8). The contraceptive device 100 is pushed along the guide wire 30 by the cannula 10 to the correct implantation site several millimeters from the tubal ostium 62 (see FIG. 9). The proper placement of the contraceptive device 100 can be guided by fluoroscopic or ultrasonic image guidance or via the working channel of a hysteroscope with a video capability to that site. To assist in guidance of the delivery cannula 10 using fluoroscopy, an appropriate marker 15 can be provided at the distal end of the delivery cannula 10 that will be readily visible in fluoroscopy.

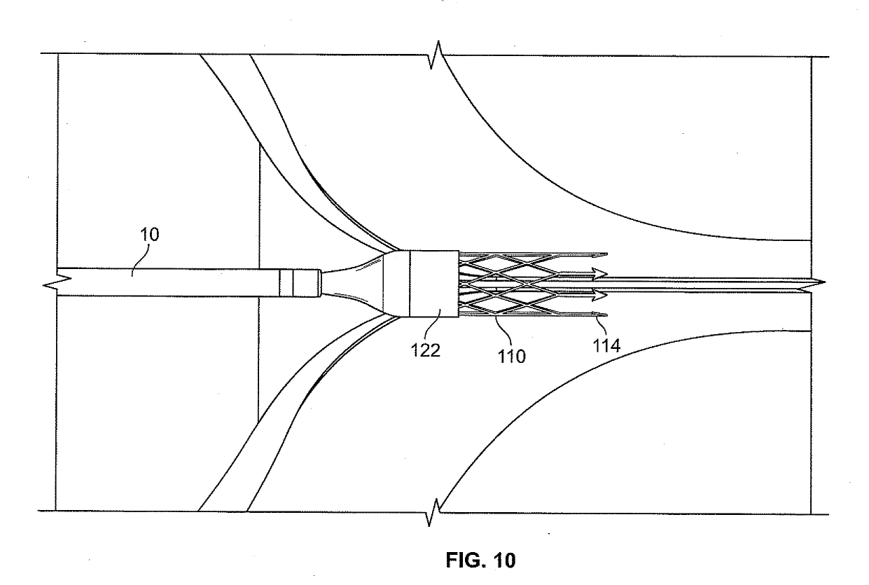

Once in the proper location for implantation, the contraceptive device 100 is pushed into the uterine cornu. As shown in FIG. 9, the barbs 114 provided at the distal end of the implant portion 110 pierce the endometrium and advance into the myometrium. The implant portion of the contraceptive device 100 circumscribes the fallopian tube opening as the contraceptive device 100 is pushed into the myometrium to the required depth, i.e. until the implant portion 110 is completely implanted into the myometrium (see FIG. 10). The depth of the implantation can be monitored and controlled by using a visual marker provided on the contraceptive device that can be seen on the fluoroscopic image, ultrasonic image or through the working channel of a hysteroscope. Alternatively, a physical stop can be provided on the contraceptive device 100 that can provide a tactile feedback via the cannula 10 to the person performing the operation. Because the metal mesh framework of the contraceptive device 100 circumscribes the fallopian tube opening, the implant does not touch the lining of the fallopian tube which might permanently damage the tube lining.

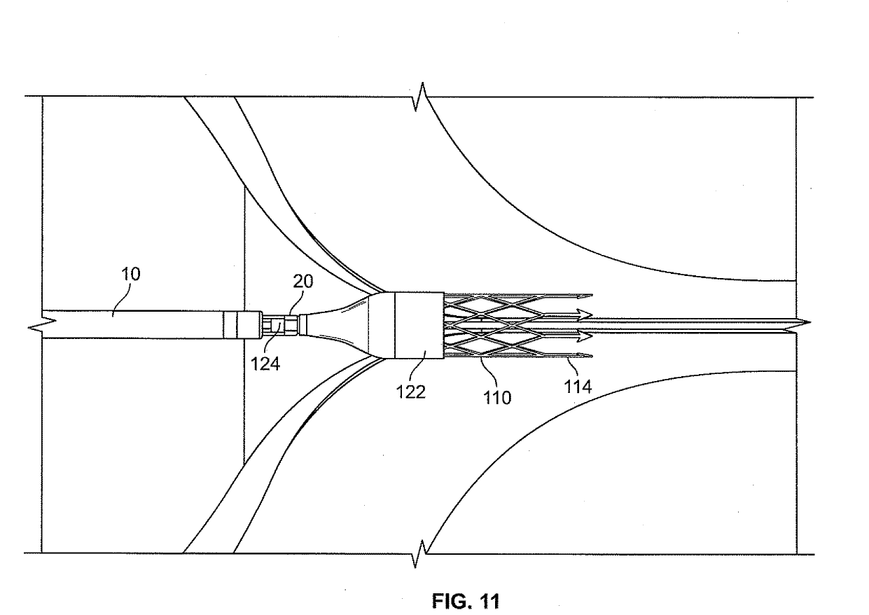

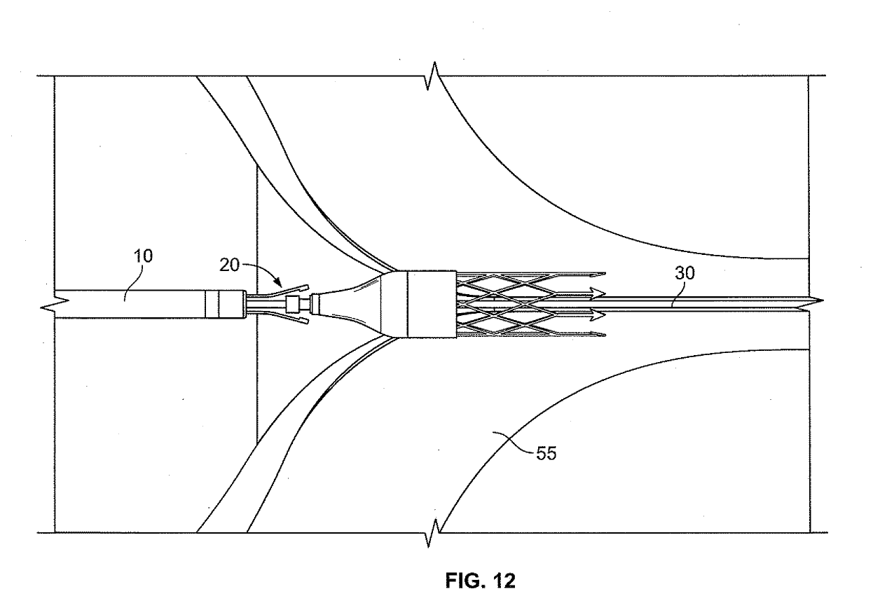

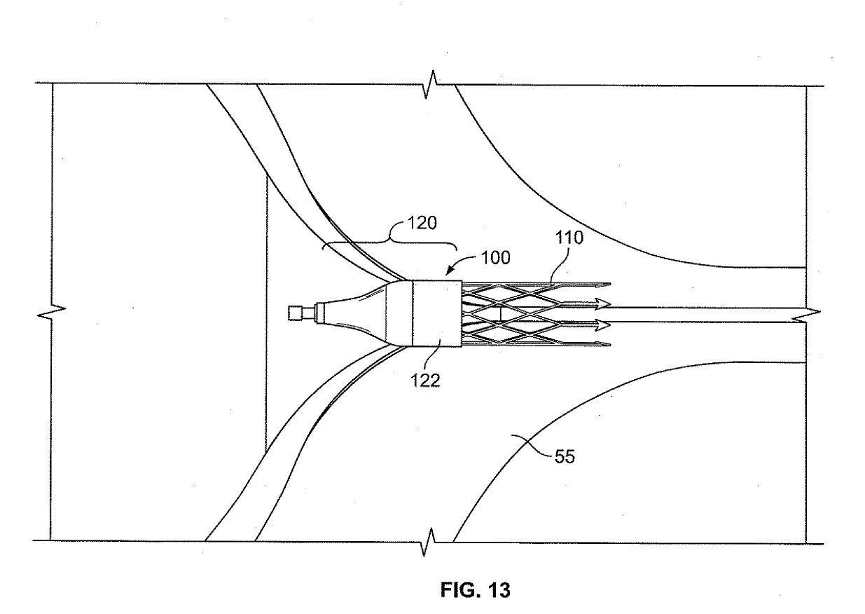

As shown in FIG. 11, once the contraceptive device 100 is implanted, the cannula 10 can be pulled back exposing the grasper 20 that is engaging the tip 124 of the cap portion 120 of the contraceptive device 100. The prongs of the grasper 20 are configured to open and release the contraceptive device 100 when the cannula 10 is pulled back sufficiently (see FIG. 12). The guide wire 30 is pulled out of the fallopian tube 60 and the cannula 10 is removed from the patient. FIG. 13 shows the contraceptive device 100 in its implanted state. The implant portion 110 is fully embedded into the myometrium 55 and the impermeable membrane 122 of the cap portion blocks the opening into the fallopian tube 60.

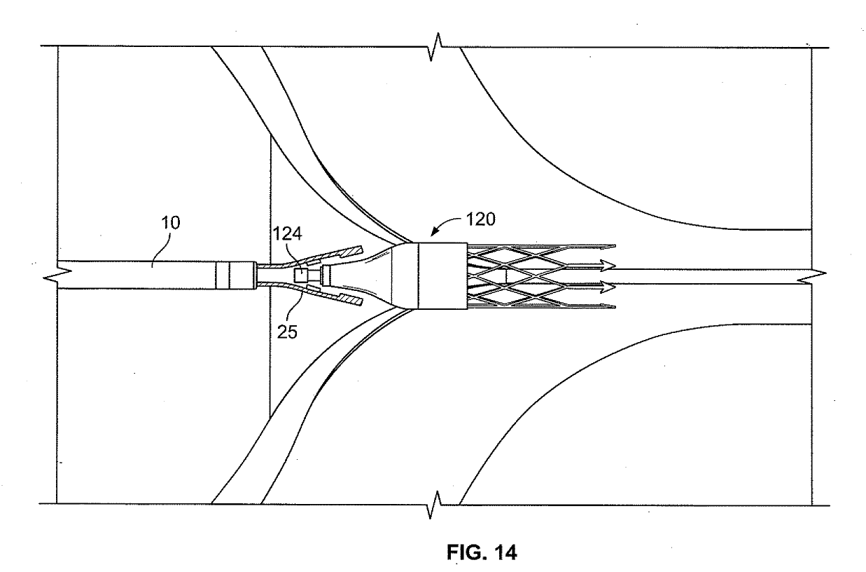

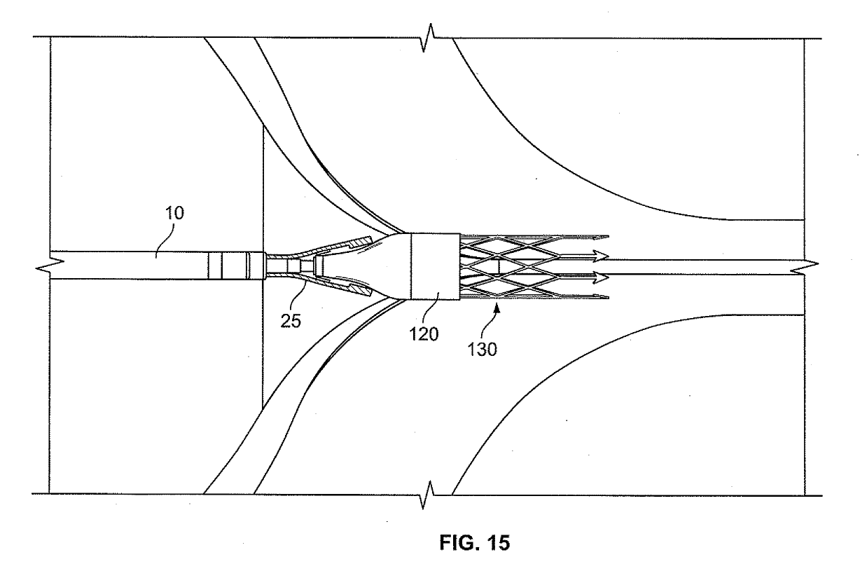

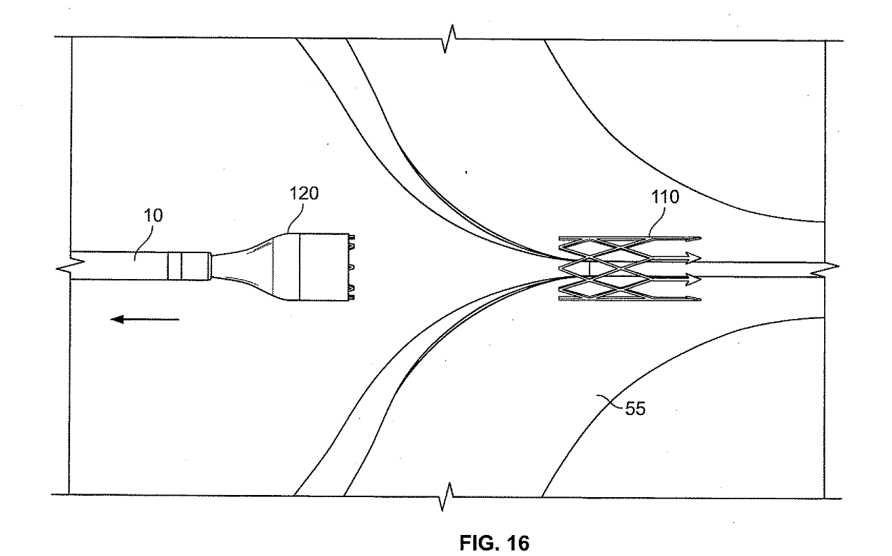

Referring to FIGS. 14-16, the procedure for removing the cap portion 120 of the contraceptive device 100 in order to reverse the sterilization will now be described. To remove the cap portion 120, the cannula 10 with a removing clamp 25 is transcervically inserted into the uterus and guided to the contraceptive device 100. The removing clamp 25 opens sufficiently wide to fit over the removable cap portion 120 as shown in FIG. 14. The cannula 10 is pushed distally toward the implanted contraceptive device 100 thus closing the removing clamp 25 around the removable cap portion 120 laterally squeezing and deforming the cap portion 120 when fully engaged as shown in FIG. 15. The deforming of the removable cap portion 120 and thus the deformation of the metal mesh framework causes the break-away zone 130 between the removable cap portion 120 and the implant portion 110 to break separating the removable cap portion 120 from the permanently implanted implant portion 110 as shown in FIG. 16. The removed cap portion 120 can now be removed transcervically along with the cannula 10. Because the implant portion 110 is completely imbedded within the myometrium there is no sign of the implant after the cap portion 120 is removed. This prevents any chance of cornual pregnancy after the reversal procedure that may be caused by ovum getting caught on a protruding piece of the implanted metal mesh.

Alternatively, the reversal of sterilization by removal of the removable cap portion 120 may be accomplished by the use of a suitably designed fine semi flexible grasping hysteroscopic accessory instrument. The semi-flexible grasping hysteroscopic accessory equipment will be similar to the removing clamp 25 except that it will be inserted into the uterus directly through the working channel of a hysteroscope rather than being inserted first through a cannula 10. The removable cap portion 120 of the contraceptive device 100 can be removed using a small diameter (5 mm or less) office based hysteroscopic system with a narrow gauge working channel to accommodate the fine removal instrument.

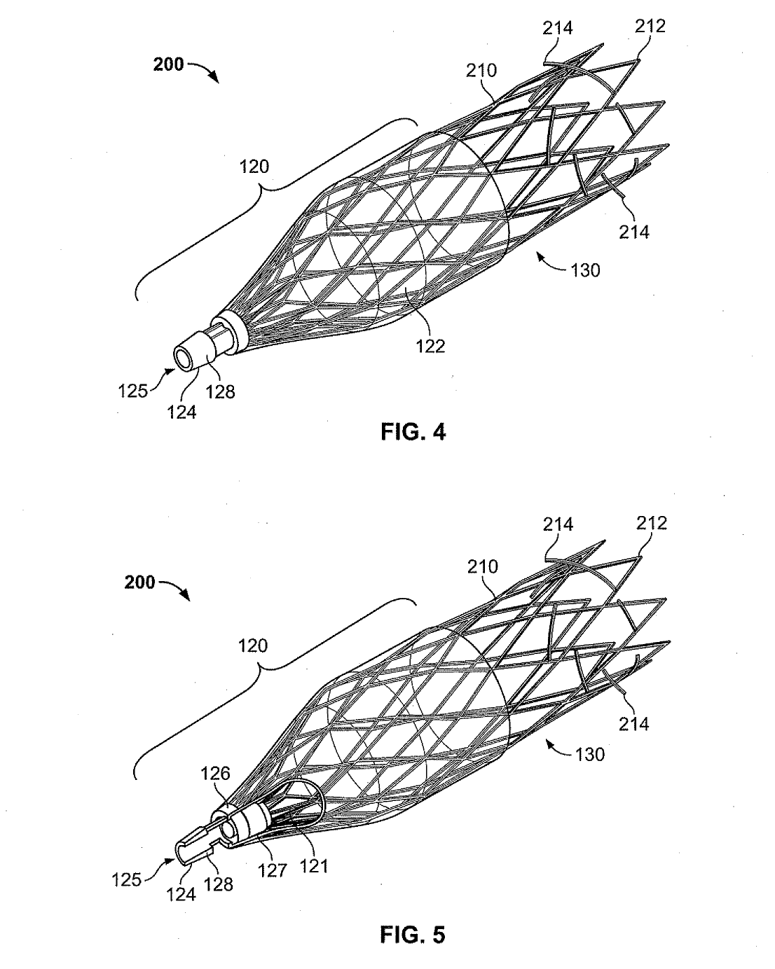

Referring to FIGS. 4-5, a contraceptive device 200 according to another embodiment is disclosed. The contraceptive device 200 has a removable cap portion 120 that is same as the contraceptive device 100. However, the distal end of the implant portion 210 is configured with a mesh pattern that forms a serrated teeth-like structure 212 as shown. This serrated teeth-like structure 212 allows the contraceptive device 200 to be implanted into the myometrium by twisting or screwing the device into the myometrium. The implant portion 210 is also provided with barbs 214 that opens up at about 45° angle in proximal direction to help fixate the contraceptive device 200 after being screwed into the myometrium. The procedure for implanting the contraceptive device 200 is similar to the procedure described above for the contraceptive device 100 with the difference being that after the contraceptive device 200 is guided to the implant site, it is twisted into the myometrium rather than being pushed straight into the myometrium. In this embodiment, the Nitinol metal mesh may have a double helicoidal mesh pattern to provide the metal mesh structure more rigidity when being twisted into the myometrium. Similar to the contraceptive device 100 of FIGS. 2-3, the contraceptive device 200 is also provided in collapsed compact configuration in the cannula 10 before deployment.

In another embodiment, the metal mesh structure of the implant portion 110 can be configured to be compressed in the longitudinal direction when being pushed against the uterine cornu. The compression of the mesh can impart more rigidity to the contraceptive device, which will facilitate driving the barbs 114 into the myometrium for implantation.

The contraceptive device's 100, 200 dimensions and configuration will take into account the relevant anatomical dimensions of the region of the uterine cornu and utero tubal junction, as well as the proportions, architecture and physical and biological properties of the different tissues. The configuration of the contraceptive device 100, 200 could be substantially conical or cylindrical in shape or a combination thereof as in the embodiments 100 and 200 discussed herein. Such a configuration will allow an adequate distance between the lumen of the utero tubal junction and the contraceptive device to ensure subsequent fibrous reaction to the metal mesh structure of the implant portions 110, 210 does not adversely affect in any significant way the utero tubal unit.

According to another aspect of the present disclosure, implantation of the contraceptive device should preferably take place within the first 9 days of the menstrual cycle but after cessation of menstrual flow. The patient should avoid sexual intercourse during the cycle of placement and should utilize a reliable primary form of contraception immediately thereafter. Such contraception should continue until a suitable imaging system has confirmed satisfactory placement and fixation of the device with no evidence of leakage after an acceptable length of time has elapsed from placement.

Based on the features of the embodiments of the method disclosed herein, further variations will now become apparent to persons skilled in the art. All such variations are considered to be within the scope of the appended claims and the scope of the appended claims are not to be limited to the particular examples of embodiments discussed herein. For example, the impermeable membrane 122 is not limited to the PTFE embodiment but can be made of any material that is impermeable to ovum and spermatozoa and having other suitable physical characteristics discussed herein. The collapsible and self-expandable shape-memory metal mesh structure of the contraceptive device is not limited to any one particular mesh structure or pattern.

What is claimed is:

1. A contraceptive device for reversibly occluding a patient's fallopian tube, comprising:
an implant portion configured for implanting into the patient's uterine myometrium in a uterine cornu and circumscribing the fallopian tube's opening; and
a cap portion removably connected to the implant portion, the cap portion having an impermeable membrane substantially impermeable to the passage of reproductive cells, wherein when the implant portion is implanted into the uterine myometrium near the fallopian tube opening circumscribing the fallopian tube opening, the cap portion occludes the fallopian tube.

2. The device of claim 1, wherein the cap portion is removably connected to the implant portion via a break-away zone.

3. The device of claim 1, wherein the implant portion and the cap portion comprise an expandable shape-memory metal material.

4. The device of claim 3, wherein the expandable shape-memory metal material is configured in a mesh structure.

5. The device of claim 3, wherein the implant portion and the cap portion are configurable in an expanded state when the implant portion is implanted into the uterine myometrium.

6. The device of claim 1, wherein the implant portion comprises a plurality of longitudinally oriented barbs for fixating the device in the uterine myometrium.

7. The device of claim 3, wherein the cap portion is removably connected to the implant portion by weakened joints between the cap portion and the implant portion, wherein the weakened joints are breakable when the cap portion is squeezed in a lateral direction to thereby separate the cap portion and the implant portion.

8. The device of claim 3, wherein the implant portion and the cap portion are fabricated as a single metal mesh having weakened joints between the cap portion and the implant portion, wherein the weakened joints are breakable when the cap portion is squeezed in a lateral direction separating the cap portion and the implant portion.

9. The device of claim 1, wherein the cap portion and the implant portion comprise separate expandable shape-memory metal meshes removably connected to each other by a mechanical fit.

10. The device of claim 1, wherein impermeable membrane is made from PTFE polymer.

11. The device of claim 1, wherein the cap portion further comprises a passageway for receiving a guide wire.

12. The device of claim 11, wherein the cap portion further comprises a one-way sealing member that seals the passageway.

13. The device of claim 11, wherein the passageway is provided by a cannulated tip and the impermeable membrane is attached to the cannulated tip.

14. The device of claim 13, wherein the impermeable membrane is attached to the cannulated tip by a locking ring.

15. The device of claim 13, wherein the impermeable membrane is attached to the cannulated tip by an adhesive.

16. A method for reversibly sterilizing a patient by occluding the patient's fallopian tube to prevent passage of reproductive cells, the method comprising:
providing a contraceptive device in a collapsed configuration inside an inner lumen of a delivery cannula, the contraceptive device comprising an implant portion configured for implanting into the patient's uterine myometrium in a uterine cornu and circumscribing the fallopian tube's opening; and a cap portion removably connected to the implant portion, the cap portion having an impermeable membrane substantially impermeable to the passage of reproductive cells, wherein a guide wire extends through the contraceptive device;
advancing the guide wire into a fallopian tube;
advancing the delivery cannula's distal end to the uterine cornu in proximity of a tubal ostium region;
advancing the contraceptive device out from a distal end of the delivery cannula to achieve an expanded configuration;
aligning the contraceptive device whereby the implant portion circumscribes the fallopian tube's opening; and
implanting the implant portion of the contraceptive device into the tubal ostium region's myometrium, whereby implant portion is substantially implanted into the myometrium and the fallopian tube's opening is occluded by the cap portion of the contraceptive device.

17. The method of claim 16, wherein the implant portion of the contraceptive device further comprises a plurality of barbs for securing the implant portion in the myometrium and implanting the implant portion into the tubal ostium region's myometrium comprises axially pushing the contraceptive device against the uterine cornu and driving the barbs into the myometrium.

18. The method of claim 16, wherein the implant portion of the contraceptive device further comprises a plurality of barbs for securing the implant portion in the myometrium and implanting the implant portion into the tubal ostium region's myometrium comprises twisting the contraceptive device against the uterine cornu and driving the barbs into the myometrium.

19. A method for reversing sterilization of a patient by occlusion of the patient's fallopian tube, the method comprising:
transcervically inserting a cannula in proximity of a tubal ostium region where a contraceptive device has been implanted, said contraceptive device comprising an implant portion and a cap portion removably connected to the implant portion;
positioning the removing clamp over the cap portion of the contraceptive device;
deforming the cap portion of the contraceptive device by squeezing the cap portion with the removing clamp, thereby separating the cap portion from the implant portion, the implant portion being implanted in the tubal ostium region's myometrium tissue; and
transcervically removing the cap portion.
